(19) United States
(12) Patent Application Publication
(10) Pub. No.: US 2015/0021878 A1
(43) Pub. Date: Jan. 22, 2015

(54) PORTABLE CARDIOPULMONARY SUPPORT CART SYSTEMS

(71) Applicant: Variety Children's Hospital d/b/a Miami Children's Hospital, Miami, FL (US)

(72) Inventors: Redmond Paul BURKE, Miami, FL (US); Robert Laurence HANNAN, Pinecrest, FL (US); Jorge Wilfredo OJITO, Miami, FL (US); Edwit George TIRONI, Weston, FL (US)

(21) Appl. No.: 14/334,989

(22) Filed: Jul. 18, 2014

Related U.S. Application Data
(60) Provisional application No. 61/847,801, filed on Jul. 18, 2013.

(51) Int. Cl. A61B 19/02; A47B 47/00; B60D 1/52

(52) U.S. Cl. CPC A61B 19/0248; B60D 1/52; A47B 47/00; A61B 2019/025
USPC 280/491.5; 280/47.34; 211/85.13

(57) ABSTRACT

Portable support cart systems are provided. The support cart can include a trailer-hitch-type connection that can attach to a movable bed, such as a gurney, for easily and conveniently transporting the cart system together with the bed. Also provided are detachable frames for transporting medical support components, as well as methods of using the same. The detachable frame can be detachably mounted on a portable cart or a movable bed, such as a gurney.

### CROSS-REFERENCE TO A RELATED APPLICATION

This application claims the benefit of U.S. Provisional Application Ser. No. 61/847,801, filed Jul. 18, 2013, which is incorporated herein by reference in its entirety.

### BACKGROUND

In patient care settings, support carts are often used to facilitate procedures by providing a portable mechanism to supply tools. For example, a hospital support cart may be used to transport equipment for a surgery or related procedure. Existing support carts have large footprints and take up significant space in patient care environments. Additionally, existing support carts typically need to be transported separately from the patient.

Conventional cardiopulmonary bypass (CPB) is a procedure that uses an extracorporeal blood circuit that is coupled between arterial and venous cannulae and includes at least a venous drainage line and an arterial return line, a blood reservoir, one pump to propel the blood in the circuit and multiple pumps to recover shed blood from the operative field and return it to the circuit via the reservoir, an oxygenator, bubble traps and or blood filters, blood flow probe, blood gas analyzers, blood transporting tubing or “lines,” ports, and valves interconnecting these components. A support cart may be used during such a procedure.

### BRIEF SUMMARY

The subject invention provides carts and cart systems that can be used in a patient care setting, as well as frames having support cart components and trailer-hitch-type connections for carts and cart systems. The patient is, preferably, a human patient and can be a pediatric patient. Advantageously, the frame and the trailer-hitch-type connection can, individually or together, allow for easy, safe, and convenient transport of medical support components in a patient care setting.

Carts, frames, and trailer-hitch-type connections of the subject invention can be easily deployed and used in tight spaces, for example in a Cathlab, an ambulance, a helicopter, or other vehicle. Such carts, frames, and trailer-hitch-type connections can be particularly advantageous in providing support for rescue patients.

In one embodiment, a medical support cart system includes a cart base having wheels and a detachable frame on which at least one medical support component is disposed. The detachable frame includes a frame base and a framework protruding from the frame base, and the detachable frame is configured to detachably mount on the cart base. The detachable frame can have a small footprint, e.g., less than 5 square feet.

In another embodiment, a detachable frame for use with a plurality of medical support components includes a frame base, a framework protruding from the frame base, and the plurality of medical support components. The detachable frame is configured to detachably mount on a portable cart. The detachable frame can have a small footprint, e.g., less than 5 square feet.

In yet another embodiment, a method of transporting a plurality of medical support components includes disposing the plurality of medical support components on a detachable frame and transporting the detachable frame. The detachable frame includes a frame base and a framework protruding from the frame base, and the detachable frame is configured to detachably mount on a portable cart. The detachable frame can have a small footprint, e.g., less than 5 square feet.

In another embodiment, a method of transporting a medical support cart system together with a gurney includes attaching a trailer-hitch-type connection to the cart system, attaching the trailer-hitch-type connection to the gurney, and moving the cart system together with the gurney while the trailer-hitch-type connection is attached to both the cart system and the gurney. The trailer-hitch-type connection can include a rigid member.

In yet another embodiment, a trailer-hitch-type connection for use with a medical support cart system can include a rigid member, a first clamp portion at a first end portion of the rigid member, and a second clamp portion at a second end portion of the rigid member opposite from the first end portion. The first clamp portion is configured to attach to the cart system, the second clamp portion is configured to attach to a movable (e.g., gurney), and the rigid member has a curved portion proximate to the second clamp portion.

In another embodiment, a medical support cart system includes a central pole, a base plate attached to the central pole, and a trailer-hitch type connection. The trailer-hitch-type connection includes a rigid member, a first end portion attached to a portion of the cart system, a second end portion opposite from the first end portion and configured to attach to a gurney, and a hinge portion configured such that the rigid member can swing.

### BRIEF DESCRIPTION OF DRAWINGS

In order that a more precise understanding of the above-recited invention can be obtained, a more particular description of the invention briefly described above will be rendered by reference to specific embodiments thereof that are illustrated in the appended drawings. The drawings presented herein may not be drawn to scale and any reference or implication of dimensions in the drawings or the following description are specific to the embodiments disclosed. Any variations of these dimensions that will allow the subject invention to function for its intended purpose are within the scope of the subject invention. Thus, these drawings depict only specific embodiments of the invention and are not limiting in scope.

FIG. 1 shows a portable support cart according to an embodiment of the subject invention.

FIG. 2 shows a portion of a portable support cart according to an embodiment of the subject invention.

FIG. 3 shows a portable support cart according to an embodiment of the subject invention.

FIG. 4 shows a portion of a portable support cart according to an embodiment of the subject invention.

FIG. 5 shows a frame mounted on a gurney according to an embodiment of the subject invention.

FIG. 6 shows a frame mounted on a gurney according to an embodiment of the subject invention.

FIG. 7 shows a frame mounted on a gurney according to an embodiment of the subject invention.

FIG. 8 shows a frame mounted on a gurney according to an embodiment of the subject invention.

FIG. 9 shows a frame mounted on a gurney according to an embodiment of the subject invention.

FIG. 10 shows a frame mounted on a gurney according to an embodiment of the subject invention.

FIG. 11 shows a frame mounted on a gurney according to an embodiment of the subject invention.

FIG. 12 shows a frame mounted on a gurney according to an embodiment of the subject invention.

FIG. 13 shows a portion of a frame according to an embodiment of the subject invention.

FIG. 14 shows a portable support cart according to an embodiment of the subject invention.

FIG. 15 shows a portable support cart according to an embodiment of the subject invention.

FIG. 16 shows a portable support cart according to an embodiment of the subject invention.

FIG. 17 shows a portable support cart according to an embodiment of the subject invention.

FIG. 18 shows a portable support cart, as well as a frame mounted on a gurney, according to an embodiment of the subject invention.

FIG. 19 shows a portable support cart, as well as a frame mounted on a gurney, according to an embodiment of the subject invention.

FIG. 20 shows a frame mounted on a portable support cart according to an embodiment of the subject invention.

FIG. 21 shows a frame mounted on a portable support cart, as well as a trailer-hitch-type connection, according to an embodiment of the subject invention.

FIG. 22 shows a frame mounted on a portable support cart and attached to a gurney with a trailer-hitch-type connection according to an embodiment of the subject invention.

FIG. 23 shows a frame mounted on a portable support cart and attached to a gurney with a trailer-hitch-type connection according to an embodiment of the subject invention.

FIG. 24 shows a frame mounted on a portable support cart and attached to a gurney with a trailer-hitch-type connection according to an embodiment of the subject invention.

FIG. 25 shows a frame mounted on a portable support cart and attached to a gurney with a trailer-hitch-type connection according to an embodiment of the subject invention.

FIG. 26 shows a portable support cart attached to a gurney with a trailer-hitch-type connection according to an embodiment of the subject invention.

FIG. 27 shows a portable support cart attached to a gurney with a trailer-hitch-type connection according to an embodiment of the subject invention.

FIG. 28 shows a portable support cart attached to a gurney with a trailer-hitch-type connection according to an embodiment of the subject invention.

FIG. 29 shows a portable support cart attached to a gurney with a trailer-hitch-type connection according to an embodiment of the subject invention.

FIG. 30 shows a portable support cart attached to a gurney with a trailer-hitch-type connection according to an embodiment of the subject invention.

FIG. 31 shows a portable support cart attached to a gurney with a trailer-hitch-type connection according to an embodiment of the subject invention.

FIG. 32 shows a portable support cart attached to a gurney with a trailer-hitch-type connection according to an embodiment of the subject invention.

FIG. 33 shows a portable support cart attached to a gurney with a trailer-hitch-type connection according to an embodiment of the subject invention.

FIG. 34 shows a portable support cart attached to a gurney with a trailer-hitch-type connection according to an embodiment of the subject invention.

FIG. 35 shows a portable support cart according to an embodiment of the subject invention.

FIG. 36 shows a portable support cart according to an embodiment of the subject invention.

FIG. 37 shows a portion of a portable support cart according to an embodiment of the subject invention.

FIG. 38 shows a portion of a portable support cart according to an embodiment of the subject invention.

### DETAILED DISCLOSURE

When the terms “on” or “over” are used herein, when referring to structures or components, it is understood that the structure or component can be directly on another structure or component, or intervening structures or components may also be present. When the terms “under” or “below” are used herein, when referring to structures or components, it is understood that the structure or component can be directly under the other structure or component, or intervening structures or components may also be present. When the term “directly on” is used herein, when referring to structures or components, it is understood that the structure is directly on another structure or component, such that no intervening structures or components are present.

The term “patient” as used herein, refers to an animal, including mammals, to which the systems and methods of the present invention are applied. Mammalian species that can benefit from the disclosed systems and methods include, but are not limited to, humans, apes, chimpanzees, orangutans, monkeys; domesticated animals (e.g., pets) such as dogs, cats, guinea pigs, hamsters; large animals such as cattle, horses, goats, sheep; and any wild animal for veterinary or tracking purposes.

The terms “surgeon” or “physician” as used in the subject invention are merely for literary convenience. The terms should not be construed as limiting. The devices, apparatuses, methods, techniques and/or procedures of the subject invention could be utilized by any person desiring or needing to do so and having the necessary skill and understanding of the invention.

Also, as used herein, and unless otherwise specifically stated, the terms “operable communication” and “operably connected” mean that the particular elements are connected in such a way that they cooperate to achieve their intended function or functions. The “connection” may be direct or indirect, physical (e.g., by wires or other physical connections) or remote (e.g., wireless).

In addition, references to “first”, “second”, and the like (e.g., first and second pressure-detector), as used herein, and unless otherwise specifically stated, are intended to identify a particular feature of which there are at least two. However, these references are not intended to confer any order in time, structural orientation, or sidedness (e.g., left or right) with respect to a particular feature.

The subject invention provides carts and cart systems that can be used in a patient care setting, as well as frames having support cart components and trailer-hitch-type connections for carts and cart systems. The patient is preferably a human patient and can be a pediatric patient. The cart can have a small cross-sectional area (of a cross-section taken parallel to the floor when the cart is in use). Advantageously, the cart has a small footprint compared to conventional carts used in patient care settings. That is, the area of the floor taken up by the cart, when it is in use, is small compared to conventional portable carts. Carts of the subject invention can be easily deployed and used in tight spaces, for example in a Cathlab, an ambulance, or other ground vehicle. Such carts can be particularly advantageous in providing support for rescue patients.

The cart can have a footprint of, for example, any of the following values, about any of the following values, no more than any of the following values, or less than any of the following values (all numerical values are in square feet): 7, 6, 5, 4.5, 4, 3.5, 3, 2.5, 2.4, 2.3, 2.25, 2.2, 2.1, 2, 1.9, 1.8, 1.7, 1.6, 1.5, 1.4, 1.3, 1.2, 1.1, 1, 0.75, or 0.5. The footprint is the cross-sectional area of the base taken in a direction that is parallel to the ground.

In one embodiment, the cart is a portable cardiopulmonary support cart. A portable cardiopulmonary support cart can provide support to a cardiopulmonary patient, such as one undergoing a cardiopulmonary bypass.

In one embodiment, the cart includes one or more of the following components: a base, a monitor, a pump, a warmer, an intravenous (IV) bag, a hook for an IV bag, a power supply (e.g., a battery-powered power supply or generator), a surge protector, and/or electrical wires or data cables. One or more monitors can be present, and at least one monitor can be a non-invasive monitor, e.g., an M3 non-invasive monitor. One or more pumps can be present, and at least one pump can be a blood pump. One or more warmers can be present, and at least one warmer can be a blood warmer. One or more IV hooks can be present to easily hang an IV bag.

In one embodiment, the electrical components present on the cart (e.g., one or more monitors, pumps, and/or warmers) can be operably connected to each other. In a further embodiment, the electrical components can be connected to each other by wires, e.g., electrical wires and/or data cables.

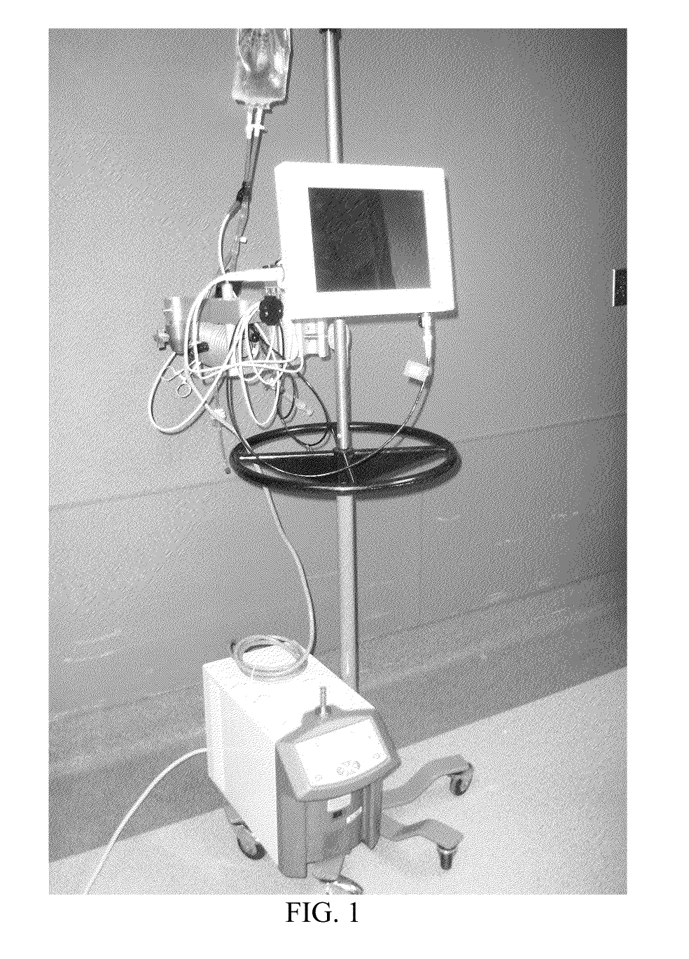

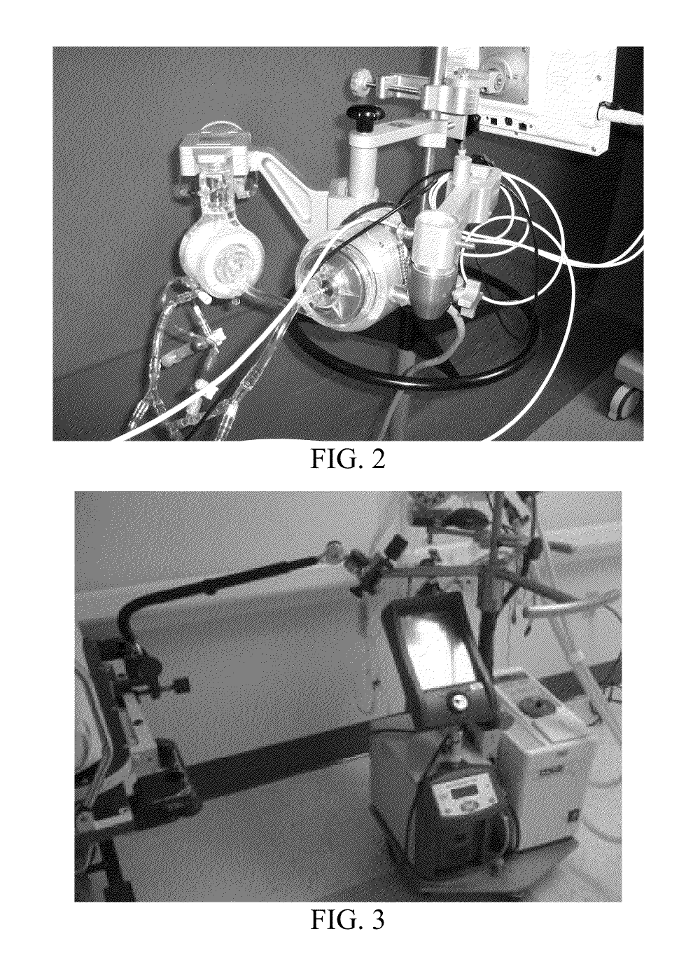

In certain embodiments, the cart has a base. In one embodiment, the base is made up of lower extending portions extending away from a central pole, as in the embodiment shown in FIG. 1. In an alternative embodiment, the base is a flat plate. The plate can be a custom plate. The custom plate can be configured such that it can easily attach to any blood pump and heater system.

In one embodiment, the cart includes a pole. The pole can be, for example, a V-pole or an IV-pole. The pole can be made of any suitable material, for example, metal, wood, or plastic. A base can be mounted to a lower portion of the pole, and other components of the cart system can be either on the base or attached to the pole. Components on the base can be either removably disposed on the base or affixed to the base, e.g., by glue, screws, other fasteners, or by interlocking parts. The base can be a plate that is attached to the pole such that it is perpendicular or approximately perpendicular to the central longitudinal axis of the pole. For example, the base can be a custom plate configured to easily attach to any blood pump and heater system.

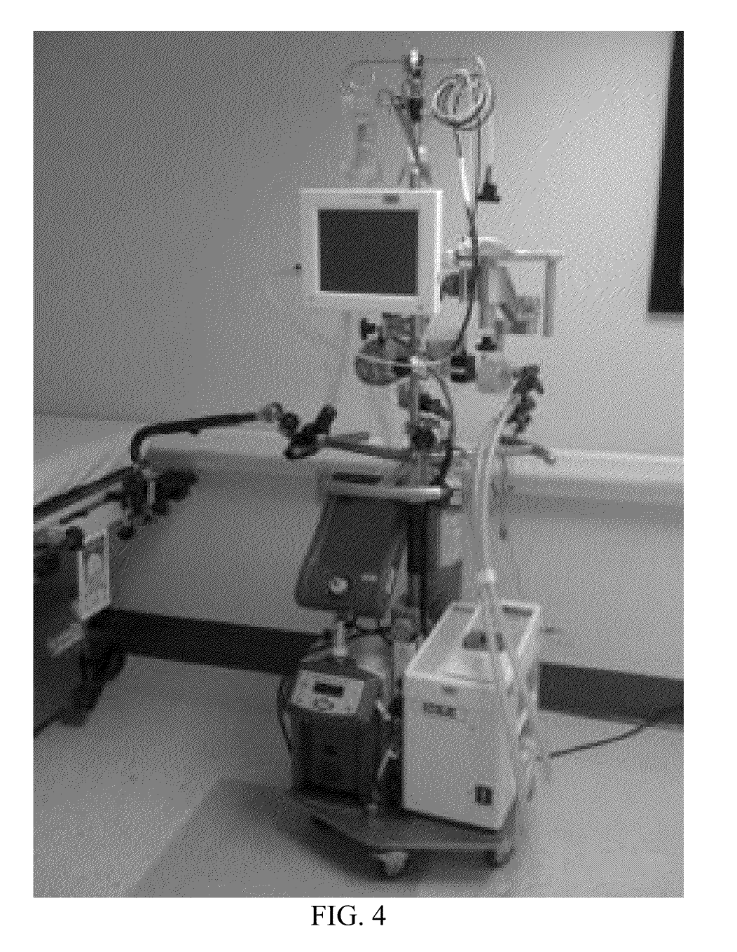

In one embodiment, the cart includes a trailer-hitch-type connection, such as an arm attachment. The arm attachment can be an adjustable articulating arm attachment. The arm attachment can be used to physically connect to a nearby bed or gurney, thereby allowing the cart to move in unison with a patient in a bed or gurney during a procedure. The arm attachment can attach to any bed typically used for patient care, for example, a Cathlab bed. This advantageously inhibits accidental separation. The arm can also be physically attached to a stretcher or gurney, increasing safety during patient transports. That is, the arm attachment can be used to quickly and easily attach the cart system to a bed or a gurney, depending on the patient's situation. Referring to FIGS. 3 and 4, an embodiment of a cart having an arm attachment is shown. In FIGS. 3 and 4, the arm can be seen as the dark member connecting the cart to the nearby bed.

In certain embodiments, the cart includes one or more handle portions. The handle portion can be, for example, a bar connected to a central pole of the cart. In one embodiment, the handle portion is a bar that encircles the pole and is connected to the pole by one or more connection portions extending from the pole, shown in FIG. 1. Though a circular bar is depicted in FIG. 1, other shapes could be used, including an oval, a square, a rectangle, a triangle, or a hexagon. In a further embodiment, the handle portion is a short bar connected to the pole by a connection portion. The cart can include more than one such handle portion. For example, three handle portions can be included as in the embodiment shown in FIG. 4. The bar and the connection portion(s) can be made of, for example, metal, wood, rubber, or plastic. The bar and the connection portion(s) can be made of the same material, though they do not need to be. The handle portion(s) can be used as hand grips and/or for holding lines and tubing.

In certain embodiments, the cart has wheels on the bottom thereof for ease of movement. The cart can have one or more sets of wheels and/or two or more individual wheels. In many embodiments, the cart has three or more wheels. The wheels can be provided individually, in sets of two or more, or some individually and some in sets. In one embodiment, the cart has a base, and wheels are provided under the base. In a particular embodiment, the base can be provided directly on the wheels. Referring again to FIG. 1, an embodiment of a cart with five wheels (four visible and one blocked) is shown. Referring again to FIG. 4, an embodiment of a cart with a base having wheels under the base is shown.

In one embodiment, the cart system includes a lifting system. The lifting system can be, for example, a hydraulic lifting system. Such a lifting system can facilitate lifting and lowering the cart system from an ambulance.

The subject invention also pertains to methods of producing and using the carts and cart systems described herein. In one embodiment, a cart system according to any of the embodiments described herein is used to provide support to a patient, for example, to provide cardiopulmonary support to a cardiopulmonary patient.

In one embodiment, the subject invention pertains to a method of producing a cart including attaching a base to a central pole. The method can further include attaching other components of the cart system, as discussed herein, to the cart system.

The subject invention also provides a frame that can be used in conjunction with support cart components. The frame can be detachable, such that it can detach from a cart while maintaining most or all of its components. In many embodiments, the frame is configured such that it can support components for use in a patient setting, such as those typically housed on a support cart. For example, the frame can have a base, on which one or more components can be disposed, and a framework. The framework can include one or more walls perpendicular to the base, though these are not required.

Components that can be supported within the frame include, e.g., one or more of the following components: a monitor, a pump, a warmer, an IV bag, a hook for an IV bag, a power supply (e.g., a battery-powered power supply or a generator), a surge protector, and/or electrical wires or data cables. One or more monitors can be present, and at least one monitor can be a non-invasive monitor, e.g., an M3 non-invasive monitor. One or more pumps can be present, and at least one pump can be a blood pump. One or more warmers can be present, and at least one warmer can be a blood warmer. One or more IV hooks can be present to easily hang an IV bag. Any electrical components present (e.g., one or more monitors, pumps, and/or warmers) can be operably connected to each other. In one embodiment, the electrical components can be connected to each other by wires, e.g., electrical wires and/or data cables.

In many embodiments, the frame is detachable and can be easily moved from a portable cart (e.g., a cart with wheels) to another structure, such as a gurney. The frame can be configured such that it can be easily picked up by one or more people and moved without losing any of the components supported within the frame. For example, one or more portions of the framework can act one or more handles.

In one embodiment, the framework of the frame includes one or more rods or bars protruding from a base or from one or more walls of the frame. The framework can include rod portions that are curved or bent at angles, thereby forming the three-dimensional structure of the frame. Components of the frame can be attached to the framework. The frame can be made of any suitable material, including but not limited to metal, wood, plastic, or rubber. The entire frame need not be made of the same material (e.g., a wooden base with metal framework). The frame can include one or more connectors for mounting to a structure (e.g., a gurney or a portable cart), though embodiments are not limited thereto. Such connectors may include, but are not limited to, straps, snap-in connectors, Velcro, or laces. In addition, or alternatively, the frame can be configured such that one or more portions of it match up with sections on the structure to which it is to be mounted, such that it locks detachably into place (e.g., can have interlocking parts). In one embodiment, the frame is mounted to a structure simply by setting the frame on the structure and allowing the frame's own weight to keep it in place.

In a preferred embodiment of the frame, the frame has a footprint that is small enough such that it can fit on a gurney with a small patient (e.g., a pediatric patient) or even between the legs of a taller patient (e.g., an adult patient).

The frame can have a footprint of, for example, any of the following values, about any of the following values, no more than any of the following values, or less than any of the following values (all numerical values are in square feet): 5, 4.5, 4, 3.5, 3, 2.5, 2.4, 2.3, 2.25, 2.2, 2.1, 2, 1.9, 1.8, 1.7, 1.6, 1.5, 1.4, 1.3, 1.2, 1.1, 1, 0.75, or 0.5.

The frame can have a height (from the base to the top of the framework) of, for example, any of the following values, about any of the following values, no more than any of the following values, or less than any of the following values (all numerical values are in feet): 4, 3.5, 3, 2.5, 2.4, 2.3, 2.25, 2.2, 2.1, 2, 1.9, 1.8, 1.7, 1.6, 1.5, 1.4, 1.3, 1.2, 1.1, 1, 0.75, or 0.5. For example, the framework of the frame can have a height of no more than 2.5 feet.

The frame can have a width (in any direction—that is, a width of the cross section of the base on which the framework is disposed) of, for example, any of the following values, about any of the following values, no more than any of the following values, or less than any of the following values (all numerical values are in feet): 5, 4, 3.5, 3, 2.5, 2.4, 2.3, 2.25, 2.2, 2.1, 2, 1.9, 1.8, 1.7, 1.6, 1.5, 1.4, 1.3, 1.2, 1.1, 1, 0.75, or 0.5. For example, the framework of the frame can have a width of no more than 3 feet.

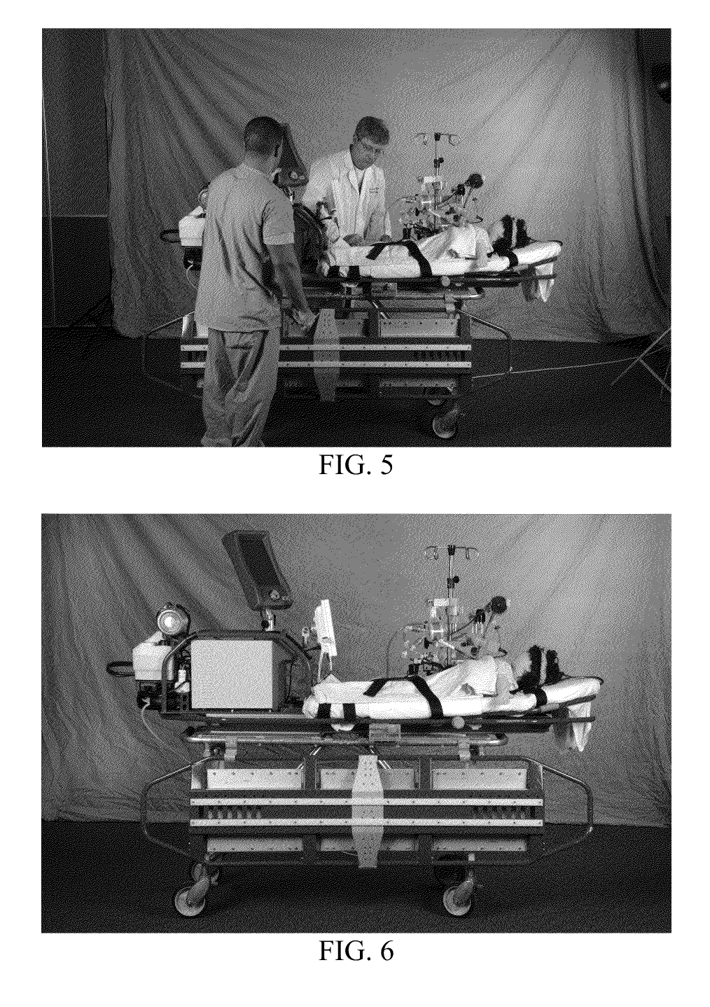

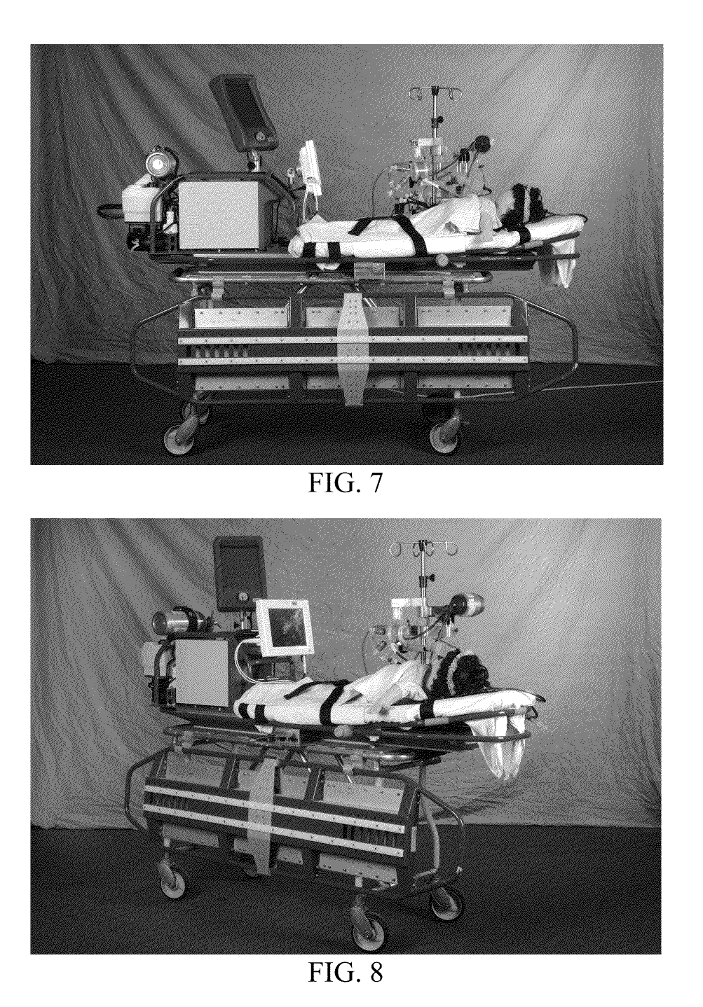

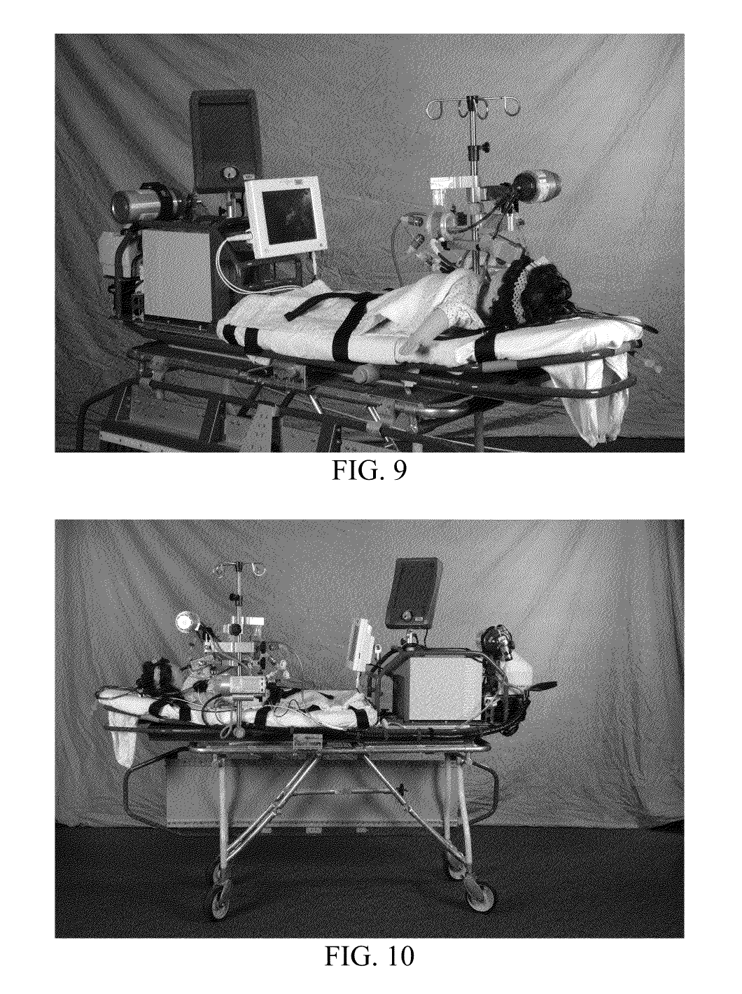

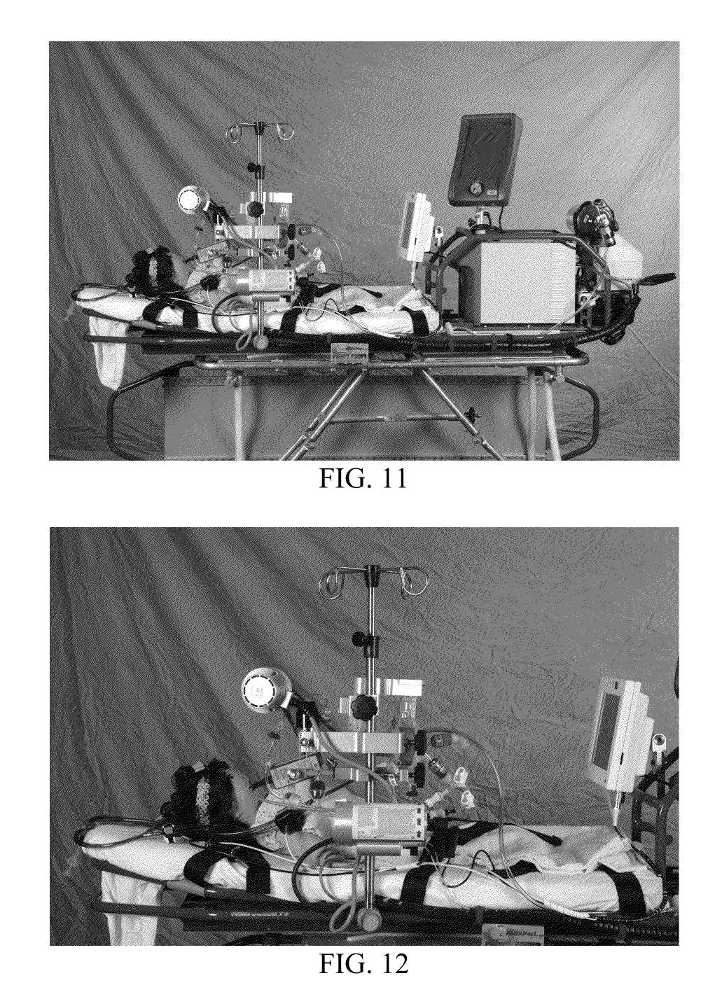

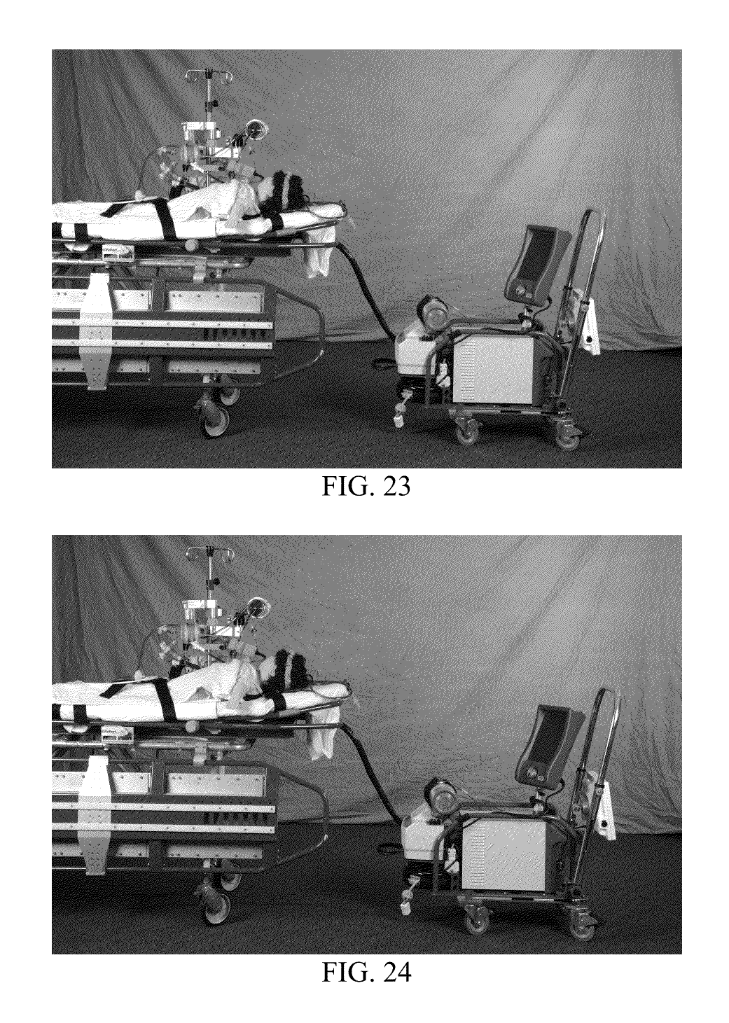

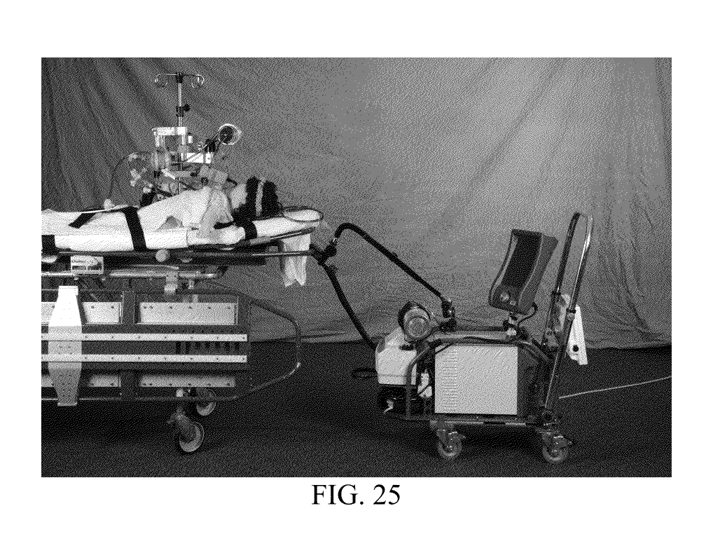

Referring to FIGS. 5-13 and 20-25, a detachable frame is shown. FIGS. 5-12 show the frame detached from a portable cart and mounted on a gurney. These figures show the frame mounted on the gurney below the feet of a prone pediatric patient (represented in the figures by a dummy). The frame has a base, on which multiple medical support components are disposed, and a framework including two parallel main rods protruding from opposite edges of the base. The rod on each side protrudes vertically (perpendicular to the base) from a corner of the base, has two curves such that it is then horizontal or approximately horizontal, has two more curves such that it is vertical again and reconnects with the base at an adjacent corner. As seen in FIGS. 8-13, the two main rods can have lateral connection portions, which are approximately perpendicular to the main rods, connecting them to each other. The framework can act as a handle to grip and move the frame.

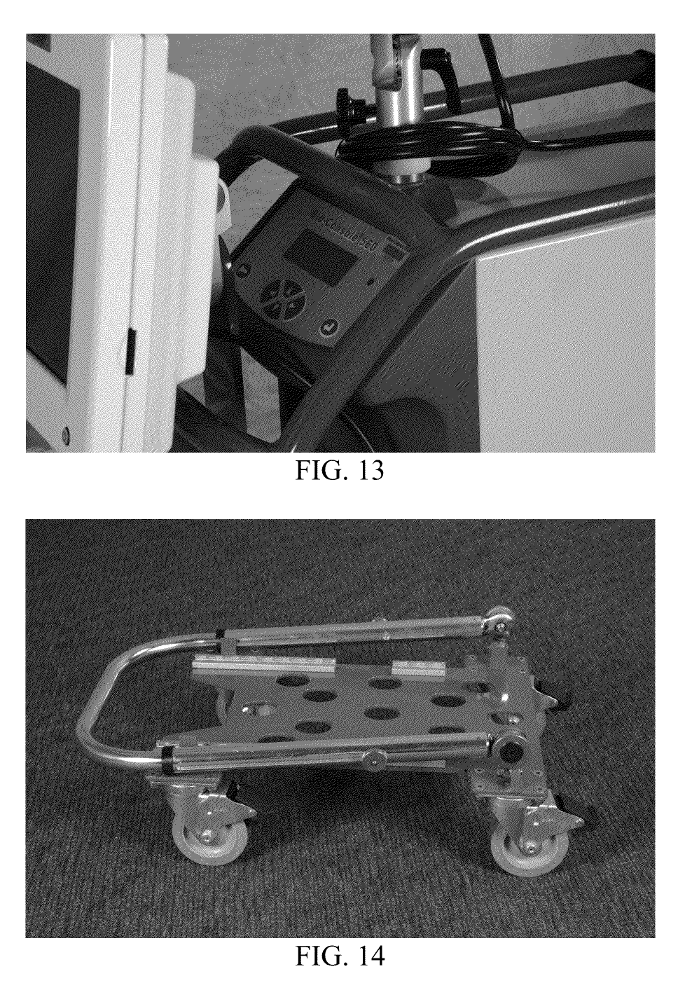

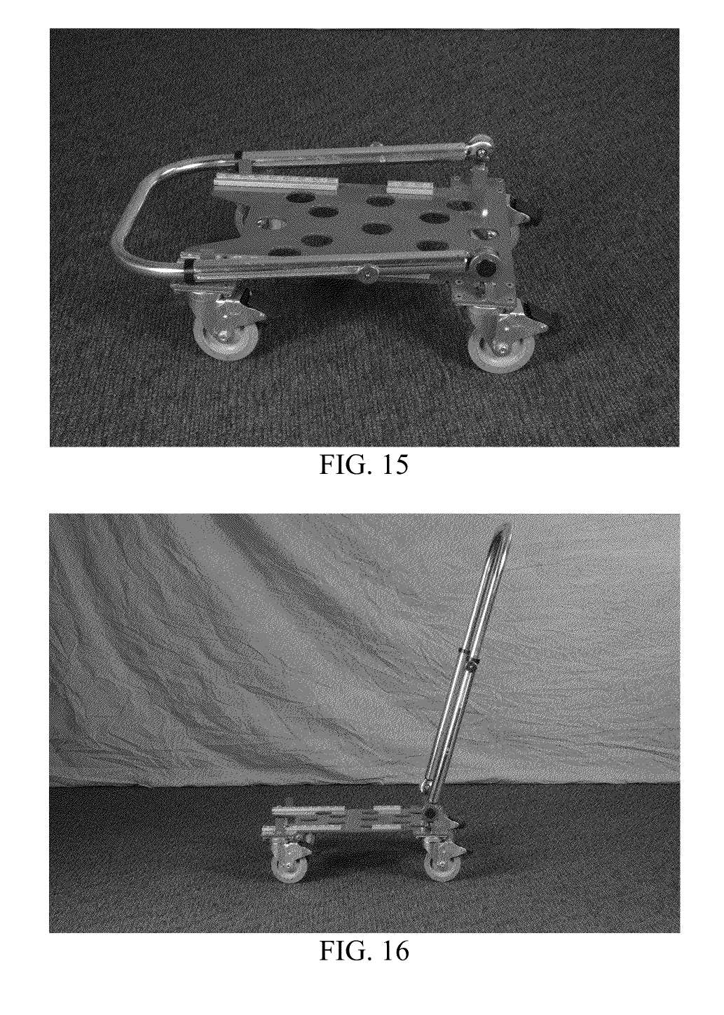

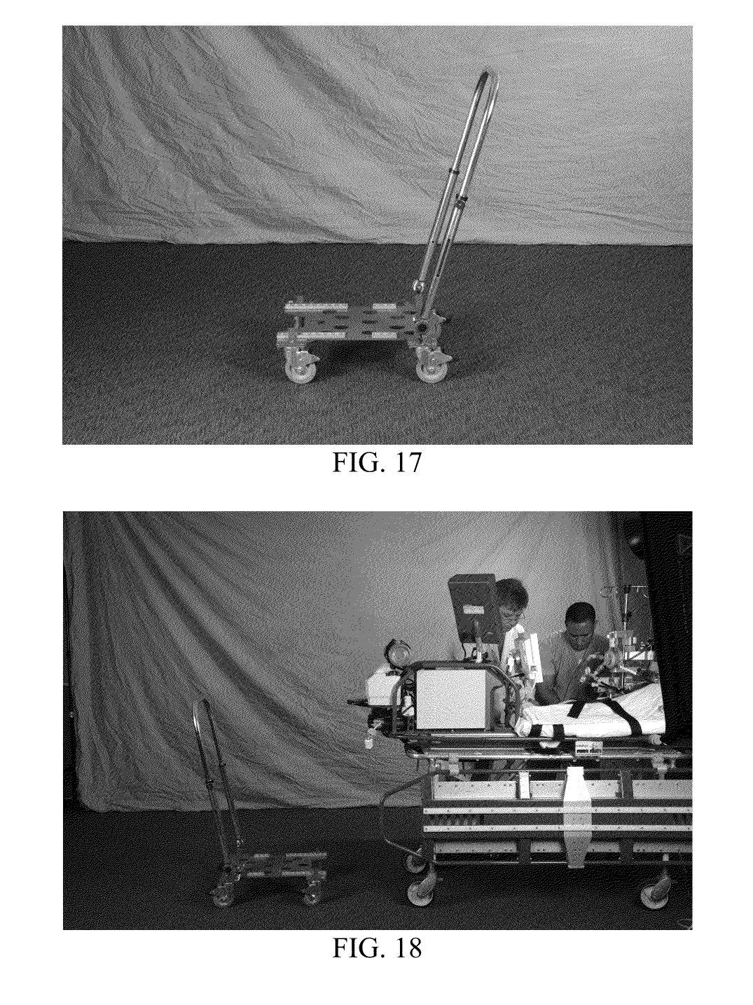

FIGS. 14-18 show an embodiment of a portable support cart that can be used with a detachable frame, according to an embodiment of the subject invention. The support cart has a base and a large handle connected to one edge of the base and configured to fold down as shown in FIGS. 14 and 15. The cart also includes wheels, with wheel brakes to help hold the cart in place. The cart shown in FIGS. 14-18 is for exemplary purposes only, and the detachable frame can be used with any portable cart that can support the frame.

In one embodiment, a method of transporting medical support components in a patient transport environment includes using a detachable frame as described herein to support the medical support components and mounting the frame on a portable structure that can also transport a patient, such as a gurney. In certain embodiments, the frame is mounted below the feet of a prone pediatric patient (or short adult patient) or between the legs of an adult patient. The frame can be easily moved from a gurney to a portable cart or similar structure.

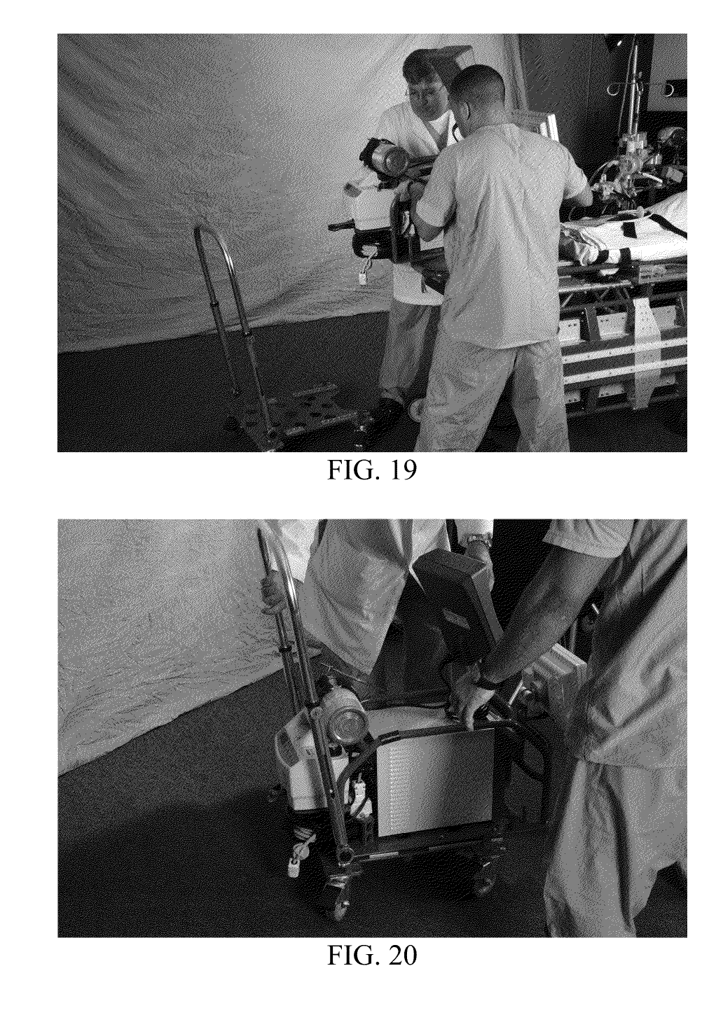

FIGS. 19 and 20 show a frame being moved from a gurney to a portable cart. The frame, gurney, and portable cart are shown for exemplary purposes only and should not be construed as limiting to the particular configurations shown. Referring to FIG. 19, the frame can be picked up from the gurney and moved towards the portable cart. Referring to FIG. 20, the frame can then be placed on the portable cart. In this way, all of the medical support components housed within the framework can be easily moved together.

Frames of the subject invention allow a cluster of medical support components to be moved together quickly and easily and also allow those same components to be easily and conveniently transported on a gurney. This is especially advantageous when space is very limited, such as during helicopter transport to and/or from a medical facility. For example, a patient can be transported in a helicopter with a frame mounted on the gurney. Then, upon arriving at the destination, the frame can be quickly and easily transferred onto a portable cart. If desired, the portable cart can be attached to the gurney using a trailer-hitch-type connection as described herein, thereby allowing the medical support components to be transported easily along with the gurney, in the case where it is desired to not have the frame on the gurney.

The subject invention also provides a trailer-hitch-type connection to connect a portable cart or cart system to a gurney or bed and methods of transporting a portable cart or cart system together with a gurney or bed using such a trailer-hitch-type connection. A trailer-hitch-type connection advantageously allows a portable cart or cart system to be easily and conveniently moved together with a gurney. The trailer-hitch-type connection can be, for example, an arm attachment as shown in FIGS. 3 and 4 as the dark member. A trailer-hitch-type connection can be made of any suitable material, including but not limited to metal, wood, plastic, and rubber.

In one embodiment, the trailer-hitch-type connection is a rigid or semi-rigid member configured to connect a portable cart or cart system to a gurney. The portable cart or cart system can be as described herein. The trailer-hitch-type connection can be, for example, a rigid rod or bar. The trailer-hitch-type connection can be fastened to the portable cart or cart system such that the trailer-hitch-type connection can swing horizontally, vertically, or both and/or can rotate. This can be accomplished through the use of, for example, a hinge portion. The trailer-hitch-type connection can be fastened to the portable cart or cart system by, e.g., one or more screws, nails, or snap connections. In many embodiments, the trailer-hitch-type connection is configured to attach to a gurney, e.g., to a portion of a bedframe of the gurney. The trailer-hitch-type connection can be configured such that it can easily attach and detach from the gurney. The attachment to the gurney can be, for example, by use of a clamp at an end of the connection and/or a curved, hook-type portion at the end of the connection.

The trailer-hitch-type connection can have a length of, for example, any of the following values, about any of the following values, no more than any of the following values, less than any of the following values, at least any of the following values, or more than any of the following values (all numerical values are in feet): 10, 9, 8, 7, 6, 5.5, 5, 4.5, 4, 3.5, 3, 2.5, 2, 1.5, 1, 0.75, or 0.5. For example, the trailer-hitch-type connection can have a length of no more than 4 feet.

In one embodiment, the trailer-hitch-type connection is permanently fastened to the portable cart or cart system. In an alternative embodiment, the trailer-hitch-type connection is configured to temporarily attach to the portable cart or cart system (e.g., through the use of a clamp). The trailer-hitch-type connection can be configured to connect directly to an existing connection (e.g., an existing clamp) on the gurney and/or on the portable cart or cart system.

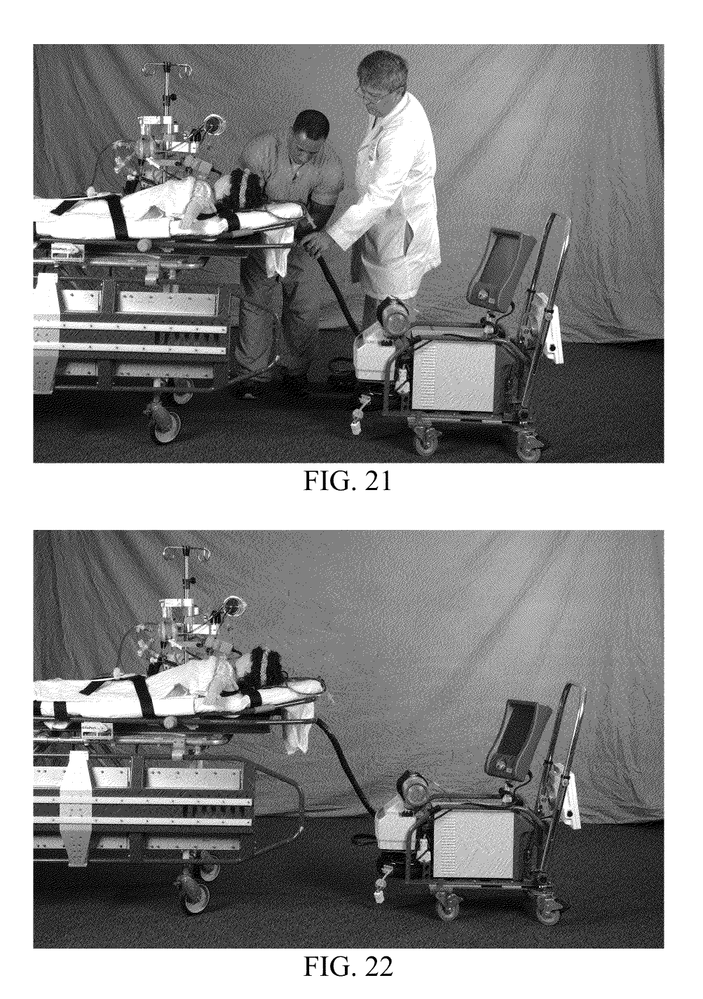

Referring to FIGS. 21-25, a portable cart having a detachable frame as described herein can attach to a gurney using a trailer-hitch-type connection as described herein. One end of the connection can be attached to the cart or the framework of the frame, and the other end of the connection can be attached to the gurney. In this way, the gurney and the portable cart can be easily and conveniently transported together.

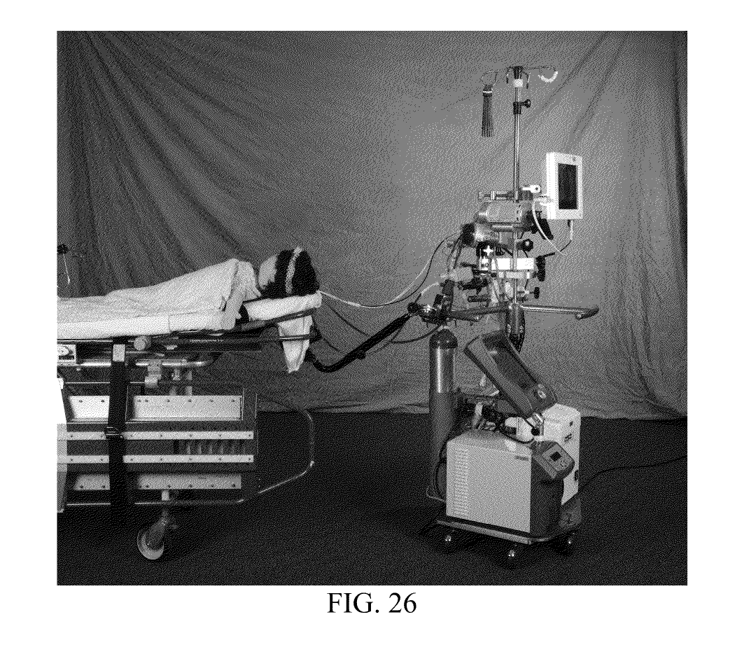

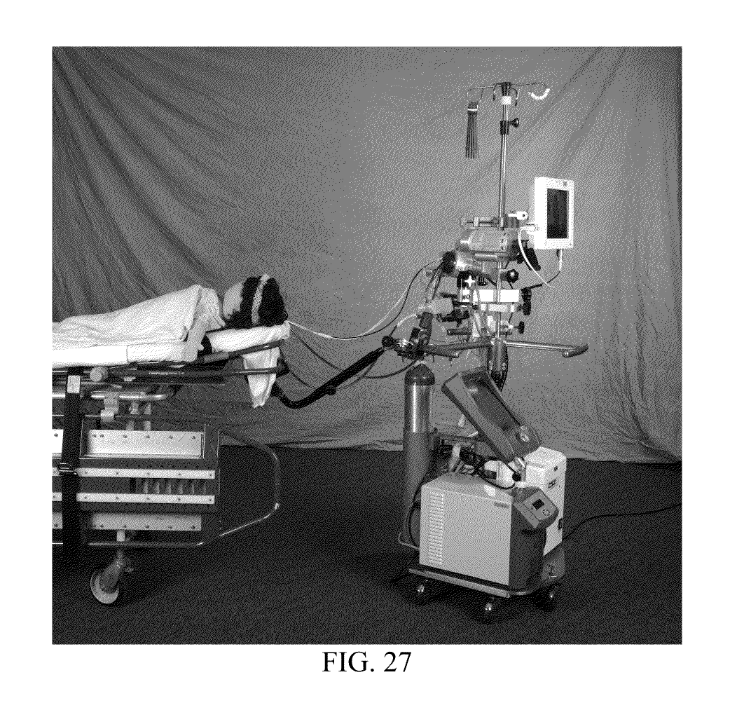

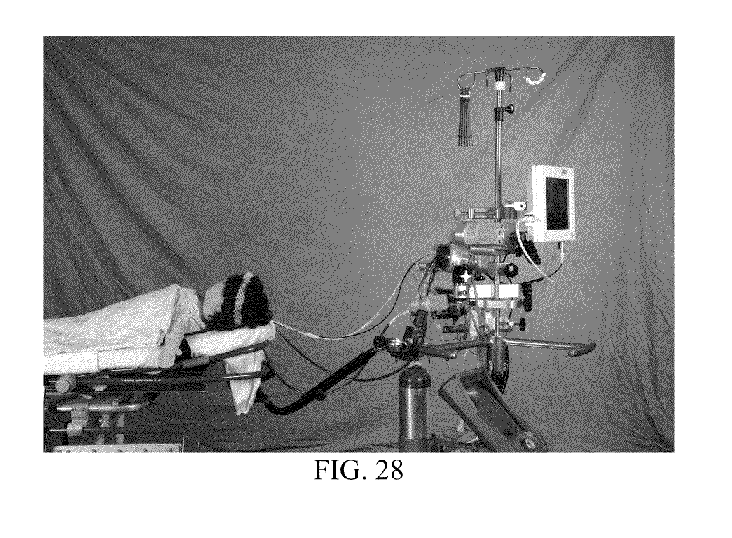

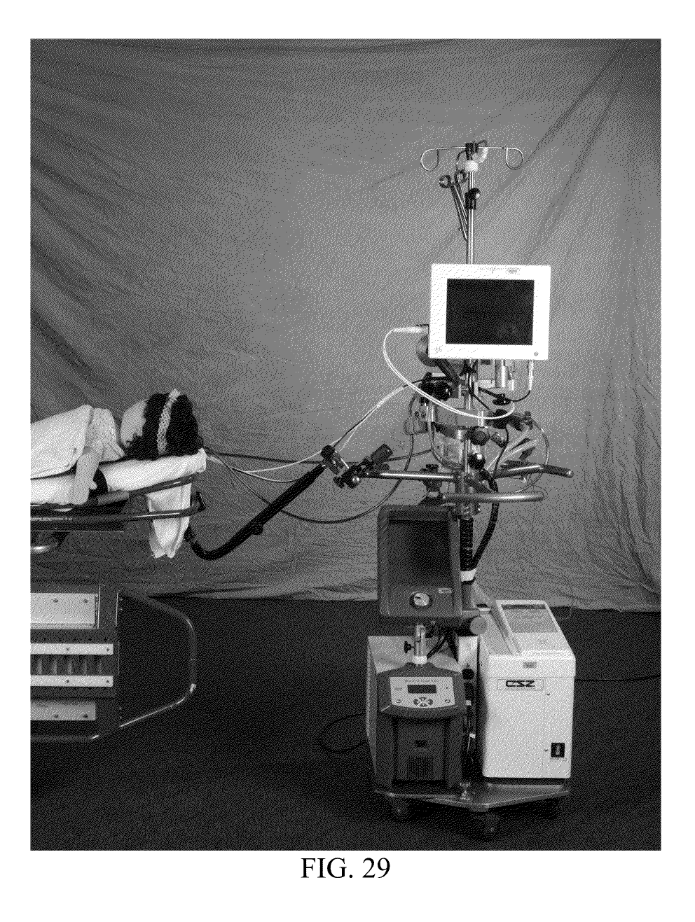

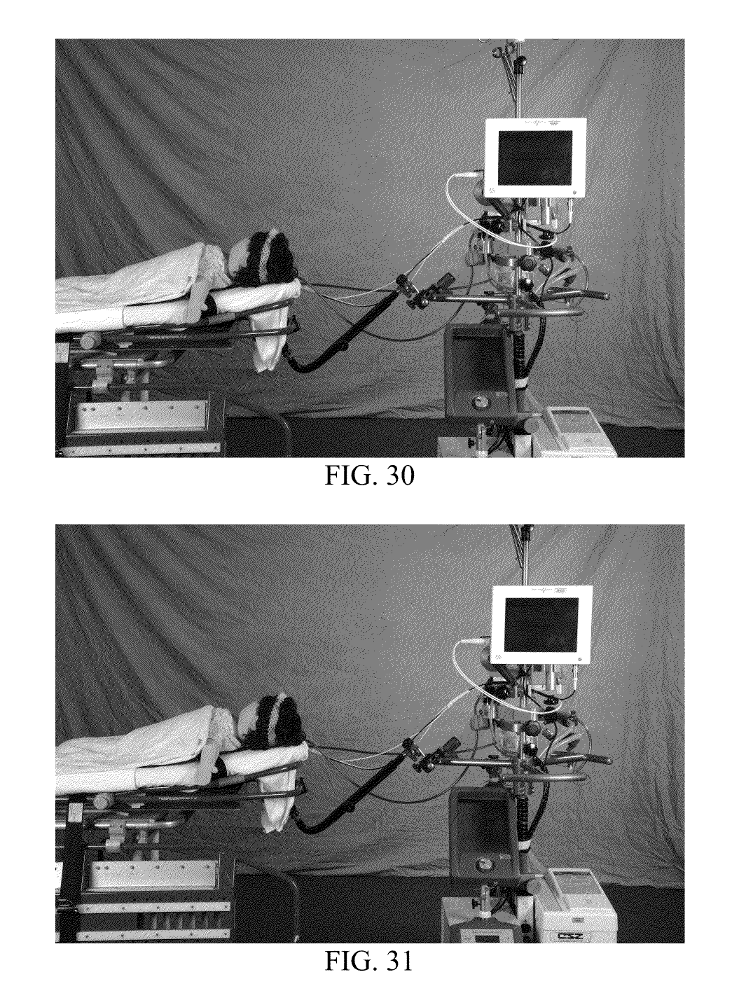

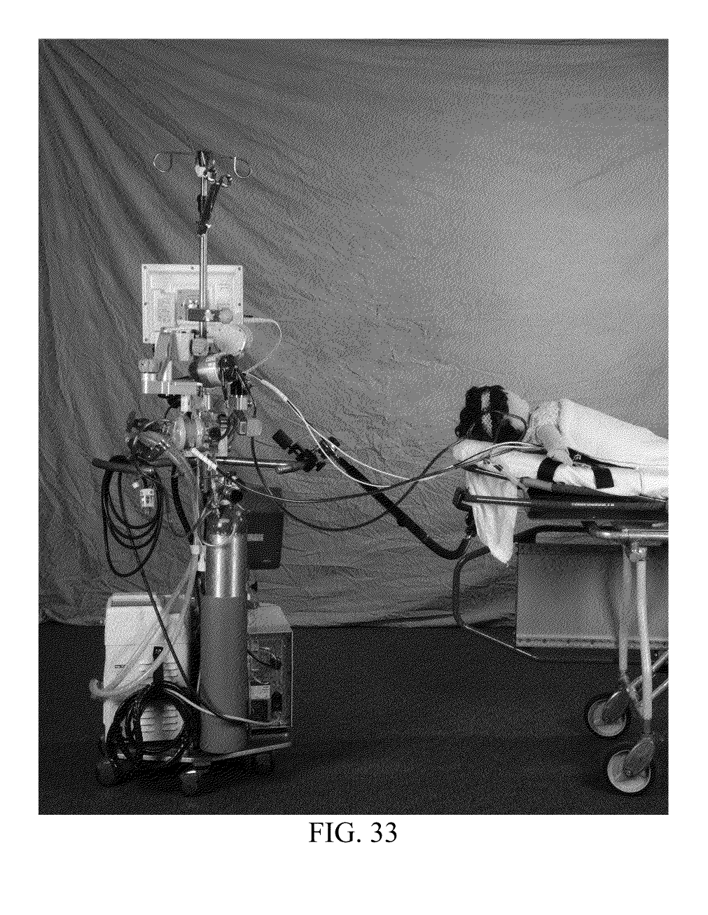

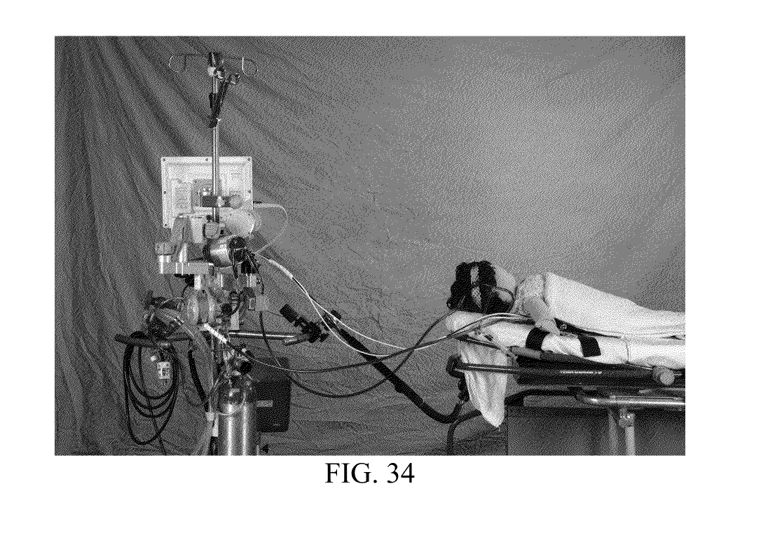

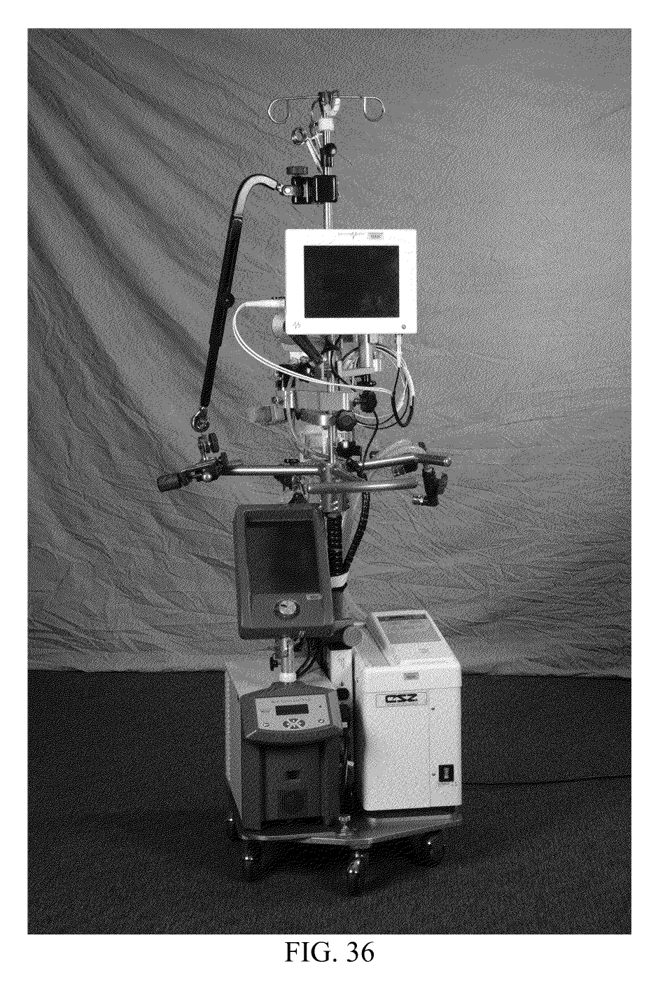

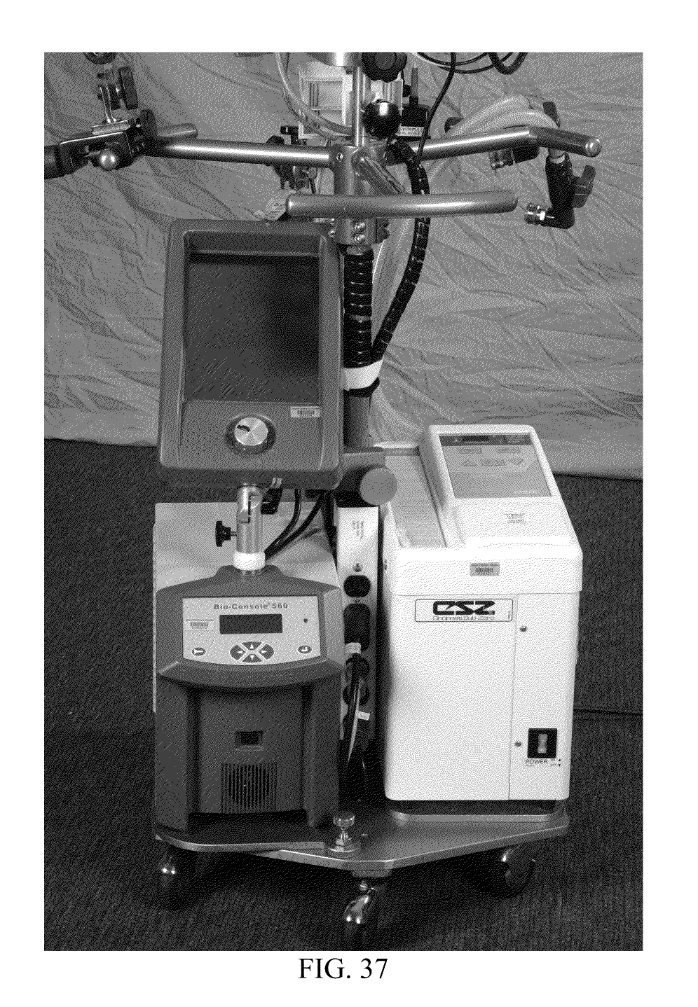

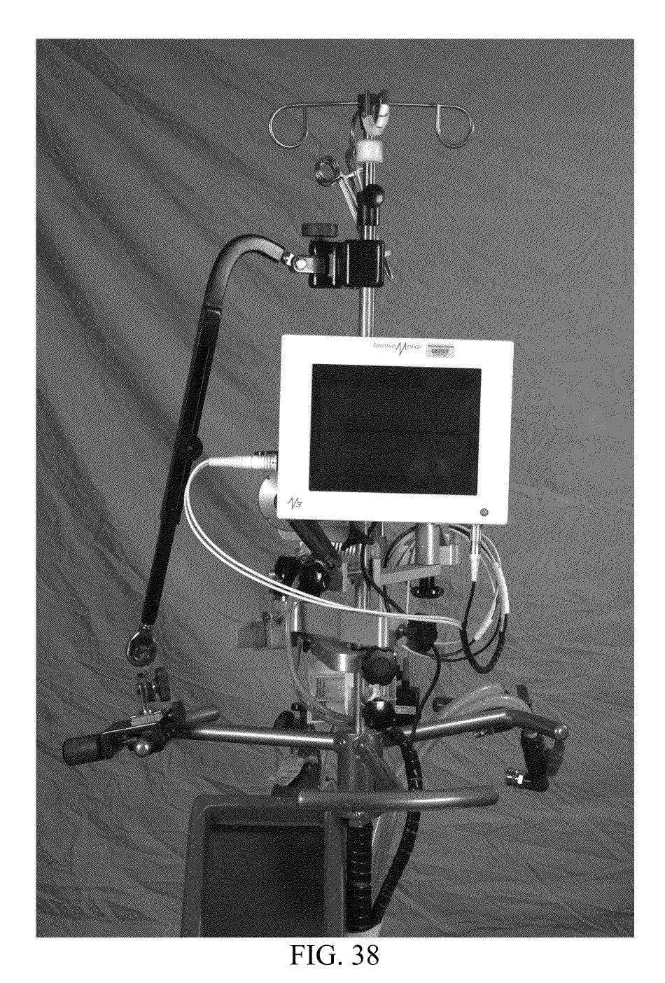

FIGS. 26-38 show examples of a trailer-hitch-type connection connecting a portable cart system to a gurney. Referring to FIGS. 26-34, the trailer-hitch-type connection can be fastened to the portable cart system via a clamp. The clamp can be, for example, configured such that it is tightened or loosened by twisting a knob provided on the clamp, though embodiments are not limited thereto. The clamp portion can also be seen in FIG. 37. The trailer-hitch-type connection can either be considered to include the clamp portion attached to the portable cart system or to not include this clamp portion.

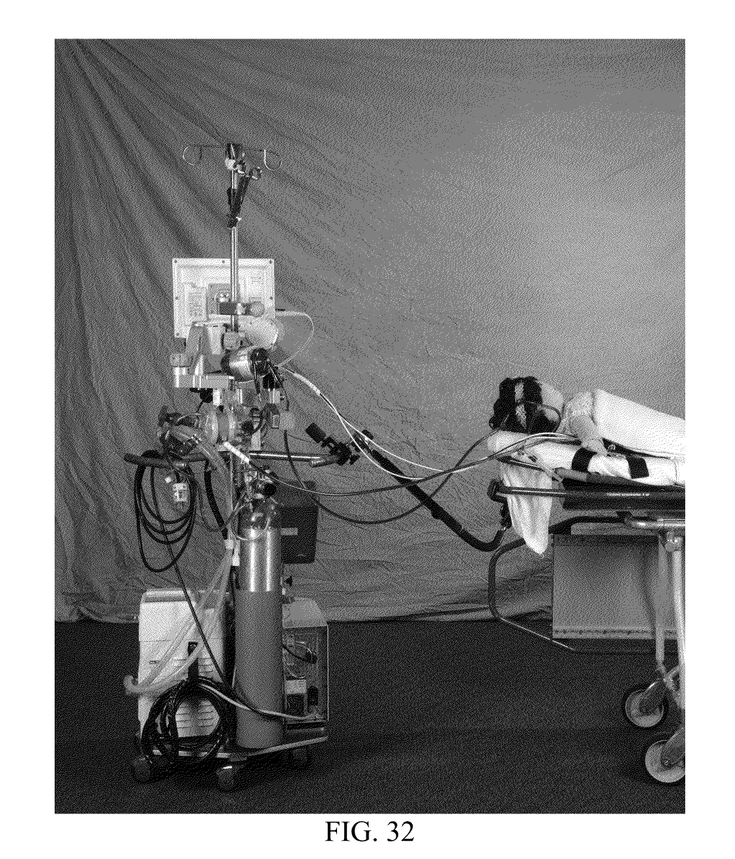

As seen in FIGS. 32-34, the trailer-hitch-type connection can also be fastened to the portable cart system via a clamp. The clamp can be, for example, configured such that it is tightened or loosened by twisting a knob provided on the clamp, though embodiments are not limited thereto. The trailer-hitch-type connection can either be considered to include the clamp portion attached to the gurney or to not include this clamp portion.

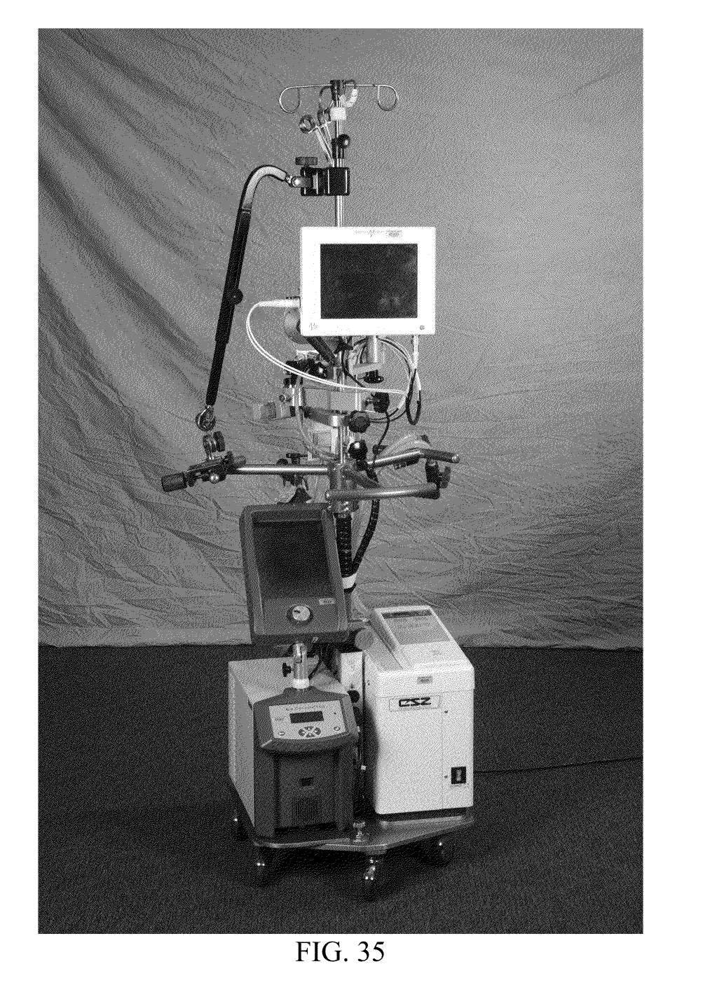

FIGS. 35, 36, and 38 show a portable cart system having a trailer-hitch-type connection. Referring to FIGS. 35, 36, and 38, the trailer-hitch-type connection can be configured such that it can swing vertically, horizontally, or both, while connected to the portable cart system. This can be accomplished through the use of, for example, a hinge portion. The trailer-hitch-type connection can also be configured to rotate, and this may also be accomplished by the use of a hinge (possibly the same hinge). These figures show the trailer-hitch-type connection positioned after having swung upwards (in an azimuthal-type direction) after being disconnected from the gurney as shown in FIGS. 26-34.

In one embodiment, the trailer-hitch-type connection can be configured such that it can be stored on the portable cart system for easy storage. Referring again to FIGS. 35, 36, and 38, the end of the trailer-hitch-type connection that can be connected to a gurney during transport with a gurney can be connected to a portion of the portable cart system (e.g., a central pole). For example, the trailer-hitch-type connection that can be connected to a gurney during transport with a gurney can be clamped to a portion of the portable cart system. This can be the same clamp that is used to connect to a gurney, or it can be a separate clamp that the trailer-hitch-type connection connects to for stable storage on the portable cart system.

In one embodiment, a portable cart or cart system is transported together with a gurney or bed using a trailer-hitch-type connection as discussed herein. The portable cart or cart system can be as described herein and may include a detachable frame as described herein, though embodiments are not limited thereto.

Following are examples that illustrate procedures for practicing the invention. These examples should not be construed as limiting.

### EXAMPLE 1

Referring again to FIG. 1, a portable cardiopulmonary support cart system is shown. The cart system includes a central pole with a base of lower extending portions extending away from the central pole. The cart system also includes an M3 non-invasive monitor, a Biomedicus blood pump, a blood warmer, and a handle portion. The handle portion is a metal bar encircling the central pole and connected thereto by multiple metal connecting portions. The monitor, the blood pump, and the blood warmer are connected to each other by data cables. An IV bag can be seen at the top portion of the image. The support cart includes five wheels, one on the underside of each lower extending portion of the base.

### EXAMPLE 2

Referring again to FIG. 4, a portable cardiopulmonary support cart system is shown. The cart system includes a central pole with a plate for a base. The plate is approximately perpendicular to the central longitudinal axis of the central pole. The cart system also includes a monitor, a blood pump, a blood warmer, and three handle portions. Each handle portion is a short metal bar connected to the central pole by a metal connecting portion. The cart system includes a trailer-hitch-type connection in the form of an adjustable articulating arm attachment which can easily physically connect to a bed (shown connected to a Cathlab bed). The monitor, the blood pump, and the blood warmer are connected to each other by data cables. An IV bag can be seen at top portion of the image. The support cart includes wheels attached to the underside of the base.

### EXAMPLE 3

Referring again to FIGS. 5-13, a detachable frame is shown. The detachable frame includes a flat base, on which multiple medical support components are disposed, and a framework including two parallel main rods protruding from opposite edges of the base. The rod on each side protrudes vertically (perpendicular to the base) from a corner of the base, has two curves such that it is then horizontal or approximately horizontal, has two more curves such that it is vertical again and reconnects with the base at an adjacent corner. The two main rods have lateral connection portions, which are approximately perpendicular to the main rods, connecting them to each other. The frame includes medical support components, including two monitors, a blood pump, and a blood warmer. One of the monitors is connected directly to one of the lateral connection portions connecting the two main rods to each other.

The detachable frame is mounted on the gurney at below the feet of a prone pediatric patient (represented by a dummy in the figures). The frame fits on the gurney along with the patient, allowing for easy transport in small spaces, such as in a helicopter.

### EXAMPLE 4

Referring again to FIGS. 14-18, a portable support cart is shown. The support cart has a base and a large handle connected to one edge of the base and configured to fold down as shown in FIGS. 14 and 15. The cart also includes wheels, with wheel brakes to help hold the cart in place. The cart can be used with the detachable frame of Example 3, such that the detachable frame can be moved from a gurney to the cart. This process is depicted in FIGS. 19 and 20.

### EXAMPLE 5

Referring again to FIGS. 21-25, a trailer-hitch-type connection is shown. The trailer-hitch-type connection is a rigid bar clamped to the cart/frame combination of Example 4 and to a gurney. The trailer-hitch-type connection can be clamped to the framework of the frame or to the support cart (e.g., to the base of the cart or to the handle of the cart).

### EXAMPLE 6

Referring again to FIGS. 35-38, a portable cardiopulmonary support cart system is shown. The cart system includes a central pole with a plate for a base. The plate is approximately perpendicular to the central longitudinal axis of the central pole. The cart system also includes a monitor, a blood pump, a blood warmer, a surge protector, and three handle portions. Each handle portion is a short metal bar connected to the central pole by a metal connecting portion. The monitor, the blood pump, and the blood warmer are connected to each other by data cables. The support cart includes six wheels attached to the underside of the base.

The cart system includes a trailer-hitch-type connection, which is a rigid metal bar. The metal bar is clamed to one of the handles, using a clamp that can be tightened or loosened using the large knob. The trailer-hitch-type connection is fastened to the clamp on the handle such that it can swing vertically (in an azimuthal-type direction) and can rotate. That is, the trailer-hitch-type connection can easily swing from a vertical position, nearly parallel to the central pole, to a horizontal position, nearly perpendicular to the central pole. The trailer-hitch-type connection is clamped to the central pole using a clamp that can be tightened or loosened using the large knob. The clamp that is clamped to the central pole can be loosened so the bar can be swung to a horizontal position and that same clamp can be clamped to a gurney for easy and convenient transportation of the cart and the gurney together.

### EXAMPLE 7

Referring again to FIGS. 26-34, a trailer-hitch-type connection is shown. The trailer-hitch-type connection is a rigid bar clamped to the portable cart system of Example 6 and to a gurney. Both clamps can be tightened or loosed using a knob. The trailer-hitch-type connection allows easy and convenient transportation of the cart system and the gurney together.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed is:

1. A detachable frame for use with a plurality of medical support components, the detachable frame comprising:
a frame base;
a framework protruding from the frame base; and
the plurality of medical support components,
wherein the detachable frame is configured to detachably mount on a portable cart, and
wherein the detachable frame has a footprint of less than 5 square feet.

2. The detachable frame according to claim 1, wherein plurality of medical support components comprises at least one blood pump, at least one blood warmer, and at least one monitor.

3. The detachable frame according to claim 1, wherein the framework comprises at least two main rods and at least one lateral connection portion connecting the two main rods to each other.

4. The detachable frame according to claim 1, wherein the frame has a height, measured from the base to the top of the framework, of no more than 2.5 feet, and
wherein the frame has a width, measured in any direction across the footprint of the frame, of no more than 3 feet.

5. A medical support cart system, comprising:
a cart base having wheels; and
the detachable frame according to claim 1,
wherein the detachable frame is configured to detachably mount on the cart base.

6. The cart system according to claim 5, wherein at least one blood pump, at least one blood warmer, and at least one monitor are disposed on the detachable frame.

7. The cart system according to claim 5, wherein the detachable frame is configured such that it can be easily gripped and transferred from the cart base to a gurney.

8. The cart system according to claim 5, wherein the framework comprises at least two main rods and at least one lateral connection portion connecting the two main rods to each other.

9. The cart system according to claim 5, further comprising a trailer-hitch-type connection configured to attach the cart system to a gurney.

10. The cart system according to claim 9, wherein the trailer-hitch-type connection comprises:
a rigid member;
a first clamp portion at a first end portion of the rigid member; and
a second clamp portion at a second end portion of the rigid member opposite from the first end portion,
wherein the first clamp portion is configured to clamp to the cart system, and
wherein the second clamp portion is configured to clamp to the gurney.

11. A method of transporting a plurality of medical support components, the method comprising:
disposing the plurality of medical support components on the detachable frame according to claim 1; and
transporting the detachable frame.

12. The method according to claim 11, wherein plurality of medical support components comprises at least one blood pump, at least one blood warmer, and at least one monitor.

13. The method according to claim 11, wherein transporting the detachable frame comprises transporting the detachable frame on a portable cart on which the detachable frame is detachably mounted or on a gurney on which the detachable frame is detachably mounted.

14. The method according to claim 11, wherein the frame has a height, measured from the base to the top of the framework, of no more than 2.5 feet, and
wherein the frame has a width, measured in any direction across the footprint of the frame, of no more than 3 feet.

15. A method of transporting a medical support cart system together with a gurney, comprising:
attaching a trailer-hitch-type connection to the cart system;
attaching the trailer-hitch-type connection to the gurney; and
moving the cart system together with the gurney while the trailer-hitch-type connection is attached to both the cart system and the gurney,
wherein the trailer-hitch-type connection comprises a rigid member.

16. The method according to claim 15, wherein the trailer-hitch-type connection further comprises:
a first clamp portion at a first end portion of the rigid member; and
a second clamp portion at a second end portion of the rigid member opposite from the first end portion,
wherein the first clamp portion is clamped to the cart system, and
wherein the second clamp portion is clamped to the gurney.

17. A trailer-hitch-type connection for use with a medical support cart system, wherein the trailer-hitch-type connection comprises:
a rigid member;
a first clamp portion at a first end portion of the rigid member; and
a second clamp portion at a second end portion of the rigid member opposite from the first end portion,
wherein the first clamp portion is configured to attach to the cart system,
wherein the second clamp portion is configured to attach to a gurney, and
wherein the rigid member comprises a curved portion proximate to the second clamp portion.

18. The trailer-hitch-type connection according to claim 17, further comprising a hinge portion attached to the first clamp portion, wherein the hinge portion is configured such that the rigid member can swing with respect to the first clamp portion.

19. The trailer-hitch-type connection according to claim 17, wherein the trailer-hitch-type connection has a total length of no more than 4 feet.

20. A medical support cart system, comprising:
a central pole;
a base plate attached to the central pole; and
a trailer-hitch type connection,
wherein the trailer-hitch-type connection comprises:
a rigid member;
a first end portion attached to a portion of the cart system;
a second end portion opposite from the first end portion and configured to attach to a gurney; and
a hinge portion configured such that the rigid member can swing.

21. The cart system according to claim 20, wherein the first end portion of the trailer-hitch-type connection comprises a first clamp portion clamped to the cart system,
wherein the second end portion of the trailer-hitch-type connection comprises a second clamp portion configured to clamp to a gurney,
wherein the hinge portion is configured such that the rigid member can swing with respect to the first clamp portion, and
wherein the trailer-hitch-type connection is configured such that the rigid member can swing to either attach to a gurney or attach to the central pole of the cart system.
